(19) United States
(12) Patent Application Publication
(10) Pub. No.: US 2010/0330648 A1
(43) Pub. Date: Dec. 30, 2010

(54) METHOD AND SYSTEM FOR PREPARING BIOMASS FOR BIOTREATMENT IN A STATIC SOLID STATE BIOREACTOR

(76) Inventors: J. Todd Harvey, Lakewood, CO (US); Glenn R. Sprenger, Golden, CO (US); Murray D. Bath, Centennial, CO (US)

Correspondence Address: Dickstein Shapiro LLP, 2049 Century Park East, Suite 700, Los Angeles, CA, 90067, US

(21) Appl. No.: 12/494,048

(22) Filed: Jun. 29, 2009

(51) Int. Cl. C12N 9/42; C12N 9/00; C12N 1/00; C12M 1/00

(52) U.S. Cl. 435/209; 435/183; 435/243; 435/289.1

(57) ABSTRACT

A method and system for preparing biomass for biotreatment in a static solid state bioreactor is performed in two stages. The first stage includes pre-mixing of the biomass with one or more reagent(s). The second stage includes the addition of a bulking agent to the pre-mixed biomass after a time sufficient for the reagent(s) to have reacted with the biomass. The second stage also includes mixing of the added bulking agent with the pre-mixed biomass to produce a biomass batch suitable for forming a static solid state particle bioreactor.

### TECHNICAL FIELD

The invention relates to treatment of biomass, and more particularly to preparation of biomass for biotreatment in a static solid state bioreactor.

### BACKGROUND

Biomass generally refers to any plant matter. This plant matter may be grown specifically for conversion to fuel, or it may be the by-product of an agricultural or industrial process which can be further utilized as fuel. Biomass may also include biodegradable wastes that can be burnt as fuel. It excludes organic material which has been transformed by geological processes into carbonaceous material such as coal or petroleum.

Production and use of biomass as a resource for fuel production is an expanding industry, with imported oil prices, sustainability, national security, and green house gas emissions being critical motivators. One path to converting biomass to biofuels comprises chemical and/or thermal preparation of the cellulosic biomass (pre-treatment), conversion of pre-treated cellulosic biomass to fermentable sugars by combinations of enzymes (saccharification), and the introduction of micro-organisms to ferment the sugars to ethanol or other synthetic fuels (fermentation).

Via this biochemical pathway, the production of a biofuel such as ethanol from cellulosic feedstocks requires the addition of one or more enzymes. The enzymes hydrolyze the complex sugars present in the biomass, converting them to simple fermentable sugars. Cellulolosic enzymes are proteins capable of breaking down cellulose in cellulosic biomass into simple sugars. Enzymes are generally specific for certain components of the cellulosic material. A fermentation agent is necessary to convert these simple sugars to ethanol. The fermentation agent is typically a yeast or microbe.

Fermentation may be broadly defined as the controlled cultivation of microorganisms for the transformation of an organic compound into a new product. Therefore, the term “fermentation” includes conventional alcohol fermentation, which is typically performed using some type of living ferment, such as yeast, and involves the enzymatically controlled anaerobic conversion of simple sugars, including those produced through saccharification, into carbon dioxide and alcohol. Depending on the organic compounds employed and fermentative microorganism(s) employed, however, a host of other fermentation products may be generated in addition to, or in the alternative to, alcohol.

Recently, conversion of biomass through fermentation into ethanol or other useful products as a replacement for fossil fuels has garnered considerable attention. Biomass for such conversion processes can be potentially obtained from numerous different sources, including, for example, wood, paper, agricultural residues, food waste, herbaceous crops, and municipal and industrial solid wastes to name a few.

For a number of reasons, biomass is an attractive feedstock for producing fossil fuel substitutes. Biomass has a smaller carbon footprint than conventional fossil fuels because it typically comes from plants that have an annual growth cycle; therefore, the carbon dioxide liberated by the combustion of the derived fuel is subsequently reused through photosynthesis by the plant's regrowth and results in no net carbon dioxide in the earth's atmosphere. Further, biomass is readily available and the conversion of biomass provides an attractive way to dispose of many industrial and agricultural waste products. Finally, biomass is a renewable resource because crops may be grown on a continuous basis, utilizing the liberated carbon dioxide each cycle.

While biomass has the potential to provide an attractive fossil fuel alternative, substantial difficulties still remain. Because the main product of the fermentation is a commodity, namely fuel, production costs must be extremely low to be competitive with other fuels. In addition, a main goal of using biomass as a fossil fuel replacement is to reduce carbon pollution. Therefore, any conversion process used should require low energy input. Because the United States alone consumes approximately nine (9) million barrels of gasoline each day, the process of creating a usable fossil fuel replacement from biomass must be scalable to be considered a meaningful alternative.

Fermentation processes can be divided into two main categories, solid state fermentation (SSF) processes and submerged liquid fermentation (SLF) processes. Solid state fermentation processes involve growth of microorganisms on moist, solid biomass particles. The spaces between the particles contain a continuous gas phase and a non-saturated water phase. Thus, although droplets of water may be present between the particles in a solid state process, and there may be thin films of water at the particle surface, the inter-particle water phase is discontinuous and most of the inter-particle space is filled by the gas phase. The majority of water in the system, therefore, is absorbed within the moist solid particles. In submerged liquid processes by contrast, particles are disposed in a continuous liquid phase.

One or more antibiotic substances are typically mixed with the biomass feedstock to suppress the proliferation of undesirable microorganisms that produce unwanted products and lower the ethanol yield.

Saccharification is the process of breaking down a complex carbohydrate (such as starch, cellulose or hemicellulose) into its monosaccharide components or sugars. Saccharification can be facilitated via the use of chemical reagents, biological agents, or combinations of these two. During alternative fuel production processes, the converted biomass is typically subjected to a saccharification process prior to or simultaneous with the fermentation process used to convert the simple sugars in the biomass, including those released through saccharification, into carbon dioxide and alcohol and/or methane.

Although SSF has been practiced for hundreds of years in the preparation of traditional fermented foods, its application to the production of fermentation products within the context of modern biotechnology has been fairly limited. This is because historically it has been notoriously difficult to control the fermentation conditions within SSF. In practice, for example, temperature control, fluid channeling, excessive pressure drop, and evaporation have posed major problems to the development of a commercially viable SSF reactor and process that is suitable for large scale, industrial applications. Thus, while the process of SSF has been practiced at small, batch, scale in the Asian food and beverage industry for hundreds of years to make soy sauce and sake and research has been conducted more recently to produce other products such as enzymes, most fermentation processes used today are still carried out in SLF processes. Indeed, all commercial fermentation processes used for producing alternative fuels that exist today employ a SLF process.

Numerous drawbacks exist with using the SLF process, however. Two principal drawbacks of SLF processes is that they tend to be capital intensive and have high operating costs, making them less than optimum for producing many fermentation products, including alternative fuels, such as ethanol, on an industrial scale and at a competitive price.

If the foregoing problems associated with SSF could be resolved, or at least sufficiently ameliorated, a commercially viable SSF bioreactor and process that is suitable for large scale, industrial applications could be achieved. Such a SSF bioreactor and process could provide several advantages over existing SLF technologies, including high product yield, low cost, ease of use, and scalability.

A wide variety of apparatus have been tried as SSF bioreactors. These apparatus fall into two main categories: static systems and stirred systems. Stirred systems have a means for mixing the biomass during the fermentation process. Stirring adds complexity and significant cost to the bioreactor. This becomes especially true for a bioreactor device that is required to be scaled up to an industrial scale to support, for example, the fossil fuel alternative market.

Static systems are sometimes used because the microorganism used in the fermentation process can not withstand the disruption caused during stirring. Various static bioreactors for SSF have been designed and used including, flasks, petri dishes, columns and trays. These designs have been mostly for laboratory use and are not effective or efficiently designed to be scaled for use at an industrial level.

One of the major problems in utilizing a static SSF bioreactor on a large scale is temperature control. The fermentation of organic compounds in general, and sugars contained or released from biomass in particular, is an exothermic reaction, generating heat in the local area of the microorganism performing the conversion. This leads to localized elevated temperatures within the biomass in the reactor. The elevated temperatures within the SSF bioreactor can result in temperatures well above the optimum for microbial growth, which in turn can inhibit the fermentation process from occurring efficiently.

When a large volume of reacting biomass is confined to a conventional solid state reactor, large temperature gradients are established within the biomass volume. This is primarily due to the fact that it is difficult to remove the localized heat uniformly from the biomass using a remote heat sink. For example, if the walls of the bioreactor are a heat sink, a temperature differential will form radially from the center outward towards the walls. With scale-up, the conduction effect of the walls of the bioreactor will have little effect on the biomass in the center of the reactor and the radial temperature gradient will increase.

Temperature gradients also form in the axial direction. As the fermentation begins, heat from the exothermic reaction tends to rise. This creates a temperature gradient in the axial direction with the top of the biomass being hotter than the bottom.

In an attempt to control the temperature of the biomass, SSF bioreactors have been designed with forced aeration. The convection and evaporation effects of the gas as it passes through the biomass have been used to reduce the temperature. Air or gas is introduced at the bottom of the biomass in the SSF and flowed to the top. By controlling the temperature and humidity of the inlet gas, the biomass in the SSF can be cooled or heated respectively.

Numerous problems exist with present forced aeration bioreactor designs. First, the gas introduced at the bottom of the reactor tends to reduce the temperature of the biomass near the bottom of the reactor, but has a lesser effect on the biomass as it passes up through the reactor. As gas is introduced, it absorbs heat from the biomass at the bottom of the reactor, which in turn raises the temperature and humidity of the gas, and makes it less effective at cooling as it passes up through the reactor. This tends to bring the temperature of the biomass at the bottom of the reactor into equilibrium with the temperature of the input gas and creates an increasing temperature gradient as the height of the biomass increases. These effects are exacerbated as the height of the SSF increases. Furthermore, the pressure drop typically increases as the height increases making forced aeration more difficult.

Because of the problems with heat removal in forced aeration SSF bioreactors, the height of the bioreactor and therefore the height of the biomass has been kept low. It has been suggested that the height of the biomass in a forced aeration SSF bioreactor should not exceed one (1) meter. See D. A. Mitchell, et al., Solid State Fermentation Bioreactors, Fundamentals of Design and Operation, Chpt. 7, 93 (2006). This creates a problem, however, because by keeping the height small, large areas are required in order to scale up existing bioreactor designs, which in many cases will be impracticable due to the availability and/or cost of the required land.

The inventors have studied the foregoing problems with static solid state bioreactors and have discovered that the above mentioned problems may be solved, or at least ameliorated in large part, by mixing the biomass feedstock to be fermented with an appropriate bulking agent in an appropriate ratio to improve the permeability of the biomass. The inventors have also discovered, however, that the manner in which the biomass feed stock and bulking agent are mixed together with reagents, such as antibiotics and saccharification agents, can have a significant impact on the overall process efficiency.

Accordingly, an object of the present patent document is to provide an improved system and method for preparing biomass for treatment in a static solid state bioreactor.

### SUMMARY

In accordance with one aspect of the invention, a method of preparing biomass for biotreatment in a static solid state bioreactor includes pre-mixing the biomass with at least one biotreatment reagent, adding a bulking agent to the pre-mixed biomass after a time sufficient for the at least one reagent to have reacted with the biomass, and mixing the added bulking agent with the pre-mixed biomass to homogenize the mixture prior to forming a static solid state bioreactor.

In accordance with another aspect of the invention, a method of preparing biomass for biotreatment in a static solid state bioreactor includes mixing biomass with at least one biodegradation reagent to form a first mixture, pre-mixing a bulking agent with at least one additional biodegradation reagent, and mixing the pre-mixed bulking agent with the formed first mixture to prepare a second mixture. The prepared second mixture is used to form a static solid state bioreactor.

In accordance with yet another aspect of the invention, a method of preparing biomass for biotreatment in a static solid state bioreactor includes pre-mixing biomass with at least one biotreatment reagent to prepare a first mixture, pre-mixing a bulking agent with at least one additional biotreatment reagent, adding the pre-mixed bulking agent to the first mixture after a time sufficient for the at least one biotreatment reagent to have reacted with the biomass, and mixing the added pre-mixed bulking agent with the first mixture to prepare a second mixture for use in forming a static solid state bioreactor. The prepared second mixture has a substantially uniform distribution of bulking agent and biomass solids.

In accordance with still another aspect of the invention, a method of preparing biomass for biotreatment in a static solid state bioreactor includes loading dried biomass into a mixing vessel, rehydrating the dried biomass load in the mixing vessel, adding a plurality of reagent solutions sequentially to the rehydrated biomass in the mixing vessel, providing sufficient mixing time for the reagent solutions and the rehydrated biomass in the mixing vessel, adding at least one bulking agent to the mixing vessel, and adding water to the mixing vessel to attain a target hydration level of mixed biomass and bulking agent solids.

In accordance with a further aspect of the invention, a system for preparing biomass for biotreatment in a static solid state bioreactor comprises a first stage which includes pre-mixing biomass with at least one biotreatment reagent, and a second stage which includes the addition of a bulking agent to the pre-mixed biomass after a time sufficient for the at least one biotreatment reagent to have reacted with the biomass. The second stage further includes mixing the added bulking agent with the pre-mixed biomass to homogenize the mixture prior to forming a static solid state particle bioreactor.

In accordance with a still further aspect of the invention, a system for preparing biomass for biotreatment in a static solid state bioreactor comprises a first stage which includes mixing biomass with at least one biodegradation reagent to form a first mixture, and a second stage which includes pre-mixing of a bulking agent with at least one additional biodegradation reagent. The second stage further includes mixing the pre-mixed bulking agent with the formed first mixture to prepare a second mixture. The prepared second mixture is used to form a static solid state bioreactor.

In accordance with a different aspect of the invention, a system for preparing biomass for biotreatment in a static solid state bioreactor comprises a first stage which includes pre-mixing biomass with at least one biotreatment reagent to prepare a first mixture, and a second stage which comprises pre-mixing a bulking agent with at least one additional biotreatment reagent, adding the pre-mixed bulking agent to the first mixture after a time sufficient for the at least one biotreatment reagent to have reacted with the biomass, and mixing the added pre-mixed bulking agent with the first mixture to prepare a second mixture for use in forming a static solid state bioreactor. The prepared second mixture has a substantially uniform distribution of bulking agent and biomass solids.

These and other aspects of the invention will become apparent from a review of the accompanying drawings and the following detailed description of the invention.

### BRIEF DESCRIPTION OF THE DRAWINGS

FIG. 1 is a schematic representation of a two-stage mixing system for preparing biomass for biotreatment in a static solid state bioreactor in accordance with an embodiment of the invention.

FIG. 2 is a schematic representation of an alternative two-stage mixing system for preparing biomass for biotreatment in a static solid state bioreactor in accordance with an embodiment of the invention.

FIG. 3 is a flowchart of a method for preparing biomass for biotreatment in a static solid state bioreactor in accordance with an embodiment of the invention.

FIG. 4 is a graph showing field test data regarding dried biomass rehydration at various moisture levels.

FIG. 5 is a graph showing field test data regarding the effect of enzyme addition on sugar extraction for various mixtures and dosages of enzymes.

FIG. 6 is a graph showing field test data regarding the effect of yeast addition on ethanol production.

FIG. 7 is a graph showing field test data regarding the impact of varying antimicrobial agent dosage on ethanol production in a SSF bioreactor.

FIG. 8 is a graph showing the effect of bulking agent volume ratio to acceptable bed height in fermentation of waste paper based on irrigation rate.

### DETAILED DESCRIPTION

Hereinafter, one or more embodiment(s) of the invention will be described with reference to the drawings. The detailed description set forth below in connection with the appended drawings is intended, however, only as a description of exemplary embodiment(s) and is not intended to represent the only embodiment(s) that may be constructed and/or utilized.

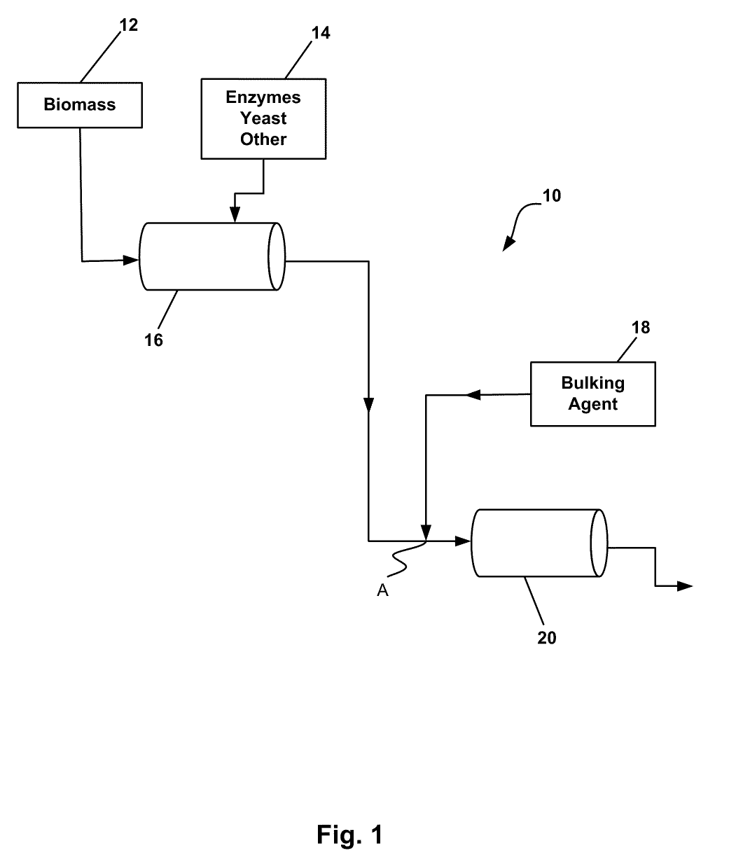

FIG. 1 schematically shows a two-stage mixing system 10 for preparing a biomass 12 for biotreatment in a static solid state bioreactor, such as a solid-state fermentation (SSF) bioreactor, in accordance with an embodiment of the invention. The biomass prepared in accordance with the methods of the present patent document are preferably subjected to simultaneous saccharification and fermentation in the static solid state bioreactors described in U.S. patent application Ser. No. 12/423,803, filed Apr. 14, 2009, and entitled “Static Solid State Bioreactor and Method of Using Same,” which is hereby incorporated by reference as if fully set forth herein.

Biomass 12 may include, for example, corn stover, corn fibers, wheat straw, wood wastes, urban wastes, switchgrass, rice straw, sugar beet pulp, citrus peels, and/or sugarcane bagasse. Other biomass matter may be used as long as such usage does not depart from the intended purpose of the invention.

The first stage involves premixing biomass 12 with one or more reagents 14 which convert the cellulosic biomass to fermentable sugars. It is desirable to have reagents 14 mixed with biomass 12 in the absence of a bulking agent so that reagents 14 attach to biomass 12 not to the bulking agent.

Reagents 14 may include enzymes, yeast or other suitable reagents such as water, recycled solution, antibiotics, nutrients and/or the like. Mixing the biomass with antibiotics at this stage, for example, would allow control of unwanted microbes. If dry biomass matter such as sugar beet pulp is to be used, adding water to the sugar beet pulp would result in the sugar beet pulp swelling extensively. Rehydrating the dry sugar beet pulp prior to stacking the same in a SSF bioreactor would allow the swelling to occur outside of the SSF bioreactor and maintain permeability. Pre-mixing of reagents with the biomass also allows for better pH control, as pH modifications done in a mixed system are much more efficient than those that must be accomplished in a static solid state bioreactor. Also, in general, a greater level of control may be achieved by mixing the components separately. The mixing vessel 16 (FIG. 1) may be a rotating drum, a screw mixer, a commercial agricultural mixer or the like.

The first stage continues until biomass 12 is sufficiently pre-mixed with reagents 14. Reagents 14 may be added to biomass 12 sequentially to optimize the pre-mixing of the feedstock. During the second stage, a bulking agent 18 is added to the pre-mixed biomass at point A (FIG. 1) and allowed to thoroughly mix with the biomass in a mixing vessel 20, as schematically shown in reference to FIG. 1. Bulking agent 18 is added to the pre-mixed biomass at this stage to enhance permeability during biotreatment in the static solid state bioreactor. The term “biotreatment” may include biodegradation, which may be generally defined as a process in which organic substances are broken down by enzymes or by living organisms.

The sequence in which the reagents are added to the biomass will allow for control in the start of the saccharification and fermentation reactions. Thus, in some embodiments, it may be desirable to mix biomass with certain reagents, such as nutrients and yeast, but delay the addition of enzymes until a significantly later point in time in order to delay the start of the saccharification and fermentation processes until a suitable time.

Bulking agent 18 adds porosity to the pre-mixed biomass, which is needed for fluid flow and permeability in the bioreactor. Bulking agent 18 may include organic materials such as almond shells (screened and unscreened) and hulls, wood chips (bark and/or wood), beet chunks, corn cobs, corn stover, orange rinds, wheat and rice straw, and/or other sized aggregates. If almond shells are to be used as a bulking agent, it may be advisable to pre-screen the almond shells to remove the fines which are presumed to contribute to lower heap permeability in the SSF bioreactor. Bulking agent 18 may also include inorganic materials such as plastic balls (spheres, bioballs), styrofoam peanuts, shredded tires, and other inert matter such as rocks and the like. An additional stage may be needed if dried biomass is being utilized to allow for rehydration of the biomass and bulking agent solids in the mixing vessel.

The prepared mixture or batch has a substantially uniform distribution of bulking agent and biomass solids. The prepared mixture may now be stacked in a static solid state bioreactor—such as that described in U.S. patent application Ser. No. 12,423,803, incorporated by reference above—under suitable (e.g., anaerobic) conditions so as to ferment the sugars to ethanol or other synthetic fuels, as needed. Further, if fermentative microorganisms were not introduced in the first stage, then such organisms may be added to the prepared mixture during or after the prepared mixture is stacked in the static solid state bioreactor.

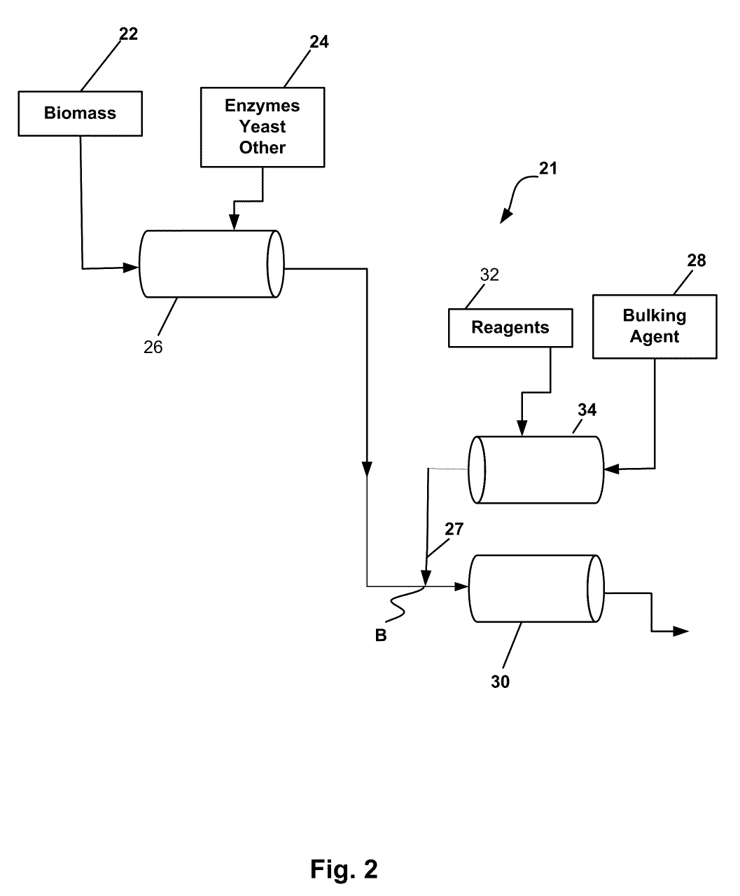

FIG. 2 schematically shows a two-stage mixing system 21 for preparing biomass 22 for biotreatment in a static solid state bioreactor in accordance with another embodiment of the invention. The first stage involves premixing of biomass 22 with one or more reagents 24 which convert the cellulosic biomass to fermentable sugars. Reagents 24 are mixed with biomass 22 in the absence of a bulking agent to ensure that reagents 24 combine with biomass 22 not with the bulking agent. Reagents 24 may be added to biomass 12 sequentially to optimize the pre-mixing of the feedstock. Reagents 24 may include enzymes, yeast or other suitable reagents such as water, recycled solution, antibiotics, nutrients and/or the like. Mixing vessel 26 (FIG. 2) may be a rotating drum, a screw mixer, a commercial agricultural mixer or the like.

The first pre-mixing stage lasts until biomass 22 is sufficiently mixed with reagents 24 in mixing vessel 26. During the second stage, a pre-mixed bulking agent 27 is added to the pre-mixed biomass at point B (FIG. 2) and allowed to thoroughly mix with the biomass in a mixing vessel 30 (FIG. 2) prior to stacking the mixed batch in a SSF bioreactor. A bulking agent 28 is mixed with one or more reagent(s) 32 in a mixing vessel 34 (FIG. 2) to produce pre-mixed bulking agent 27. Reagents 32 may include, for example, light acid for sterilization of the bulking material, or recycled solution for the purposes of rehydration. Pre-mixed bulking agent 27 is added to the pre-mixed biomass to enhance permeability of the biomass during biotreatment in the static SSF bioreactor. Pre-mixed bulking agent 27 may include one or more of the organic and inorganic materials described hereinabove. Each of mixing vessels 30 and 34, as shown in FIG. 2, may be a rotating drum, a screw mixer, a commercial agricultural mixer or the like.

The prepared mixture or batch has a substantially uniform distribution of bulking agent and biomass solids. The mixed batch may be used to form a SSF bioreactor such as that described in U.S. patent application Ser. No. 12/423,803, incorporated by reference above. The formed SSF bioreactor is utilized to ferment the separated sugars to ethanol or other synthetic fuels under suitable environmental conditions.

Referring to FIGS. 1 and 2, one continuous mixing vessel with various addition points along its axis (not shown) may be utilized instead of separate vessels 16 and 20 (FIG. 1) and vessels 26, 30 and 34 (FIG. 2), respectively, to practice the invention. Other suitable system modifications and/or configurations may be employed, as desired.

Apparent advantages of biotreatment systems 10 and 20, as generally described hereinabove, include, but are not limited to, control of reagent addition points and reduced reagent consumption, intimate mixing of the bulking agent with the biomass, control of the kinetics, as well as intimate mixing of the biomass and reagents and therefore even mass distribution in the static solid state bioreactor.

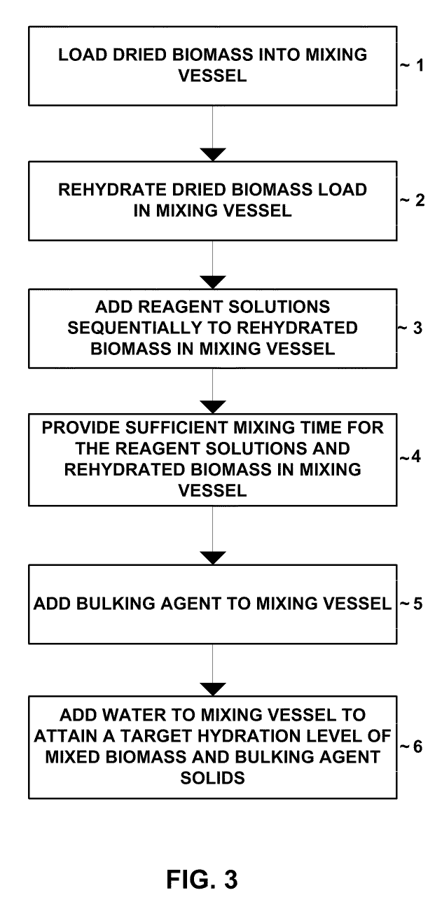

FIG. 3 is a flowchart of a method for preparing biomass for biotreatment in a static solid state bioreactor in accordance with an embodiment of the invention. Step 1 involves loading dried biomass into an appropriate mixing vessel. For example, in a field test performed by Applicant, dried sugar beet pulp (SBP) containing 9.2% moisture as received, was loaded into an agricultural feed mixer such as the 20 m³ capacity Trioliet® vertical-auger batch mixer, by a front end loader. Approximately 1,600 kg was charged for each batch. The mass was recorded by the mixer's load cell.

Step 2 includes rehydrating the dried biomass load in the mixing vessel. Specifically, in the same field test performed by Applicant, water was added to achieve a water content of 65% in the SBP. Approximately 2600 kg of water was added per batch. The quantity of water added was measured by both the mixer load cell and a flow meter in the supply line. To ensure sufficient rehydration time was provided in the feed mixer, the rate and extent of hydration of dried SBP in contact with water were investigated. It was found that dried SBP absorbs water very quickly, reaching ˜70% moisture by weight almost instantaneously. Tests were performed to measure the rehydration rate at target moisture contents between 70% and 95%.

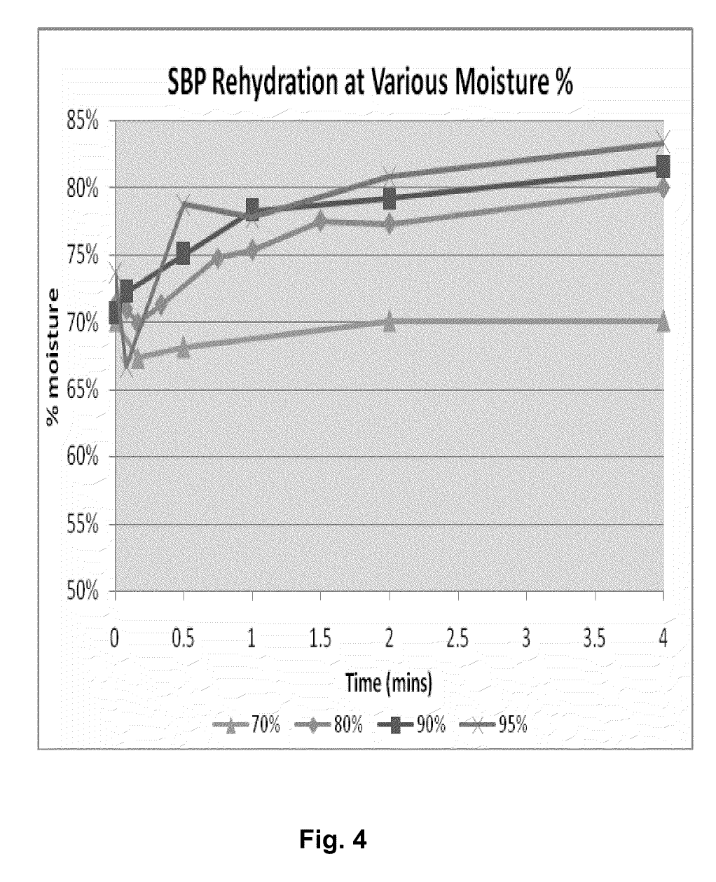

Dried SBP and water were mixed together for various times, the mixture poured over a screen to drain off excess water, and the amount of water absorbed by the SBP recorded. FIG. 4 shows that initial rehydration to between 65 and 72% moisture is almost instantaneous and that higher moisture contents require longer contact time.

Rehydration to 65% moisture was selected for the field test. It was also determined that this level of rehydration would support good enzymatic digestion.

The next step (3) deals with various reagent solutions being added sequentially to the rehydrated biomass in the mixing vessel. In this regard, as part of the same field test performed by Applicant, reagent solutions were added sequentially in the following order: (a) enzymes, (b) yeast, and (c) yeast nutrients with antibiotics. Solution volumes were measured by a totalizing flow meter.

Sugar beet pulp, like other food wastes, is low in lignin and easily attacked by enzymes with little or no pretreatment. The plant cell wall is strengthened by “cables” of cellulose called microfibrils. These cellulose microfibrils are glued together by hemicelluloses and pectin to make cell walls, the main material comprising cellulosic biomass. The enzyme cellulase breaks down crystalline cellulose into cellobiose, which is a dimer of two glucose molecules. Another enzyme, beta-glucosidase converts cellobiose into single glucose molecules (simple six-carbon sugar). A third enzyme, pectinase, converts pectin, which is one of the main polysaccharides in sugar beet pulp, into galactose (another simple six-carbon sugar), arabinose (simple five-carbon sugar), and galacturonic acid (a sugar acid). Other enzymes may include Exoglucanase 1, Exoglucanase 2 and Endoglucanase E1. Beta-glucosidase is derived from the Aspergillus niger. Exoglucanase 1 and Exoglucanase 2 are derived from the Trichoderma reesei (Hypocrea jecorina). Endoglucanase E1 is derived from the Acidothermus cellulolyticus. It will be appreciated from the teachings contained herein that a combination of any of the above listed enzymes may also be utilized to practice the invention. Specifically, three enzymes were added to the biomass in the field test to release the contained sugars: Novo 188 (beta-glucosidase), Celluclast with 5% Novo 188 (cellulase), and Pectinex (pectinase). These enzymes were procured from Novozymes Corp. of Salem, Va. as concentrated broths. The three enzymes were combined in a single 1,900 liter tank.

The yeast species Saccharomyces cerevisiae has been used for centuries to convert simple six-carbon sugars to ethanol. However, the enzymatic hydrolysis of cellulosic biomass typically results in the production of both five- and six-carbon sugars. Accordingly, if desired, a microorganism capable of fermenting five-carbon sugars may also be employed to produce ethanol from the five carbon sugars generated the saccharification process. The field test performed by Applicant, however, focused on fermenting only the six-carbon sugars (using Saccharomyces cerevisiae). Particularly, Ethanol Red™ yeast, manufactured by Fermentis® (a division of the Lasaffre Group of Milwakee, Wis.), was employed for the test.

Yeast requires nutrients for propagation. Fermaid K™ is a blended complex yeast nutrient that supplies ammonia salts (DAP), alpha amino nitrogen (derived from yeast extract), sterols, unsaturated fatty acids, key nutrients (magnesium sulfate, thiamin, folic acid, niacin, biotin, calcium pantothenate) and inactive yeast. GO-FERM™ is a natural yeast nutrient containing a balance of micronutrients. Both of these yeast nutrients used in the field tests may be purchased from Scott Laboratories of Petaluma, Calif.

Contamination in fermentation systems may lead to side reactions which produce unwanted products as well as diminish the alcohol yield. As part of the field test, bacterial control agents Lactrol® (virginiamycin and dextrose) and Nisin® (an antimicrobial peptide produced by certain strains of Lactococcus lactis) were added to the feedstock to control contamination prior to forming the SSF bioreactor.

During the field test, an agitated 380 liter tank was utilized to mix and store the nutrient and antibiotic solutions. Each tank was equipped with a 75 l/min centrifugal pump delivering to the feed mixer via a common header. The header was equipped with a water flush for cleaning after each batch. A 19,000 liter tank was used to store water for SBP rehydration and reagent make-up. A 760 l/min centrifugal pump supplied water to the mixer as well as to the reagent tanks

Step 4 requires the provision of sufficient mixing time for the various reagent solutions and rehydrated biomass described hereinabove in the mixing vessel. Specific field test mixing time data follows hereinbelow.

Step 5 involves the addition of at least one bulking agent to the batch mixture present in the vessel. Particularly, as part of the field test, screened almond shells, containing 18.9% moisture as received, were loaded into the mixer by a front end loader. Approximately 1,600 kg was charged for each batch. The mass was recorded by the mixer's load cell. Permeability of enzymatically digested SBP mixed with bulking agent was determined by subjecting the test material to a load in a compression cell and then measuring the ability of the mixture to pass the desired fluid flows. Liquid was applied to the top of the apparatus while gas (air) was fed to the chamber from below. Gas pressure build-up above 50 mm of water in the bottom of the chamber indicated unacceptable performance. In addition to its performance in load-permeability testing, local availability of the bulking agent for the field test was considered; almond shells met the required criteria. A 1:1 mass ratio of almond shells to SBP was selected for the field test. The following table shows selected results of the permeability testing on a variety of bulking agents.

| | Ratio | Degraded | |
|---|---|---|---|
| Bulking Agent | (SBP:BA) | SBP | Pass/Fail |
| Balls | 1:1 | no | Pass |
| Almond Hulls | 1:1 | no | Fail |
| Almond Hulls | 1:1 | yes | Fail |
| Almond Shells | 1:1 | no | Pass |
| Almond Shells | 1:1 | yes | Pass |
| Hulls/Shells | 1:1 | yes | Fail |
| Corn Cobbs | 1:1 | yes | Pass |
| Corn Cobbs | 2:1 | yes | Fail |
| Rice Straw | 1:1 | yes | Fail |
| Corn Stover | 1:1 | yes | Fail |
| Unsieved Almond Shells | 1:1 | no | Pass |
| Unsieved Almond Shells | 2:1 | no | Pass |
| Sieved Almond Shells (less −2 mm) | 1:1 | no | Pass |
| Sieved Almond Shells (less −2 mm) | 2:1 | no | Pass |

Step 6 includes the addition of water to the mixing vessel for the purposes of attaining a pre-set target hydration level of mixed biomass and bulking agent solids. Specifically, during the field test, water was added to hydrate the almond shells to a target moisture content of 50%. Approximately 1700 kg of water was added per batch. The quantity of water added was measured by both the mixer load cell and a flow meter in the supply line. The mixing time for each batch was approximately 16 minutes. The total batch cycle time, including the time required to discharge the mixer, was 27 minutes. The quantities of each component in each field test batch and the totals for the SSF are listed in the table hereinbelow.

| Batch # | Wt SBP (kg) | Wt AS (kg) | Water (L) | Fermaid K (g) | Yeast (kg) | Goferm (g) | Nisin (g) | Lactrol (g) | Enzymes (L) |
|---|---|---|---|---|---|---|---|---|---|
| 1 | 1644.27 | 1655.61 | 4579.77 | 111.58 | 11.40 | 68.39 | 22.80 | 28.12 | 94.82 |
| 2 | 1696.43 | 1637.47 | 4570.91 | 110.99 | 11.40 | 68.39 | 22.80 | 27.97 | 94.94 |
| 3 | 1639.74 | 1660.15 | 4590.94 | 110.49 | 11.41 | 68.45 | 22.82 | 27.85 | 94.79 |
| 4 | 1673.75 | 1642.00 | 4613.14 | 110.39 | 11.42 | 68.50 | 22.83 | 27.83 | 95.35 |
| 5 | 1644.27 | 1664.68 | 4601.87 | 110.89 | 11.36 | 68.16 | 22.72 | 27.95 | 90.17 |
| 6 | 1705.51 | 1628.40 | 4586.52 | 109.40 | 11.34 | 68.05 | 22.68 | 27.58 | 87.22 |
| 7 | 1719.11 | 1644.27 | 4547.59 | 109.69 | 11.43 | 68.57 | 22.86 | 27.65 | 88.65 |
| 8 | 1628.40 | 1569.43 | 4638.08 | 111.08 | 11.44 | 68.61 | 22.87 | 28.00 | 103.57 |
| 9 | 1628.40 | 1644.27 | 4428.27 | 104.73 | 11.39 | 68.32 | 22.77 | 26.41 | 76.01 |
| 10 | 1630.66 | 1691.90 | 4518.21 | 111.28 | 11.44 | 68.64 | 22.88 | 28.05 | 86.99 |
| 11 | 1655.61 | 1637.47 | 4507.33 | 111.08 | 11.46 | 68.75 | 22.92 | 28.00 | 87.93 |
| 12 | 1632.93 | 1635.20 | 4603.30 | 111.68 | 11.48 | 68.91 | 22.97 | 28.15 | 87.82 |
| 13 | 1623.86 | 1637.47 | 4500.28 | 110.39 | 11.40 | 68.41 | 22.80 | 27.83 | 88.05 |
| 14 | 1673.75 | 1637.47 | 4657.06 | 113.47 | 11.27 | 67.64 | 22.55 | 28.59 | 89.15 |
| 15 | 1626.13 | 1646.54 | 4610.07 | 111.08 | 11.38 | 68.27 | 22.76 | 28.00 | 88.92 |
| 16 | 1687.36 | 1646.54 | 4624.91 | 112.18 | 11.45 | 68.68 | 22.89 | 28.27 | 87.97 |
| 17 | 1651.07 | 1653.34 | 4690.53 | 123.31 | 15.23 | 91.41 | 30.47 | 31.13 | 133.09 |
| total | 28161.26 | 27932.20 | 77868.78 | 1893.70 | 197.69 | 1186.14 | 395.38 | 477.38 | 1575.45 |

Moreover, as part of the field test, four batches of yeast were made up for addition to the SBP. Each batch consisted of 51.75 kg of yeast, 311 g of GO-FERM™, 1.035 g of Lactrol™, and 103.5 g of Nisin™, added to 518 L of warm water. Dry components were pre-weighed, mixed and added to water heated to 35° C. The batch was allowed to stand for 30 minutes before addition of the appropriate volume to the first SBP batch in the mixer. The yeast addition was equivalent to 0.75% of the dry mass of the SBP. The yeast suspension also contained 600 ppm GO-FERM™, 2 ppm Lactrol™, and 5 ppm Nisin™. Enzymes were supplied as liquid broths, which were blended before addition to the SBP. The blend consisted of 232.8 L of beta-glucosidase, 1175.4 L of cellulase, and 189.9 L of pectinase. Details of the enzymes are summarized in the table hereinbelow.

| Enzyme | Beta-Glucosidase | Cellulase | Pectinase |
|---|---|---|---|
| Units | CBU/g | EGU/g | PGU/mL |
| Activity per unit | 242 | 807 | 10741 |
| Density (g/mL) | 1.244 | 1.22 | 1.182 |
| Quantity (L) | 232.8 | 1175.4 | 189.9 |

Additionally, two batches of nutrient solution were produced for addition to the SBP during the field test. Each batch consisted of 248.4 g of Lactrol™, and 993.6 g of Fermaid K, added to 378.79 L of water. Dry components were added to 35° C. water and mixed for 30 minutes before the first addition to the mixer.

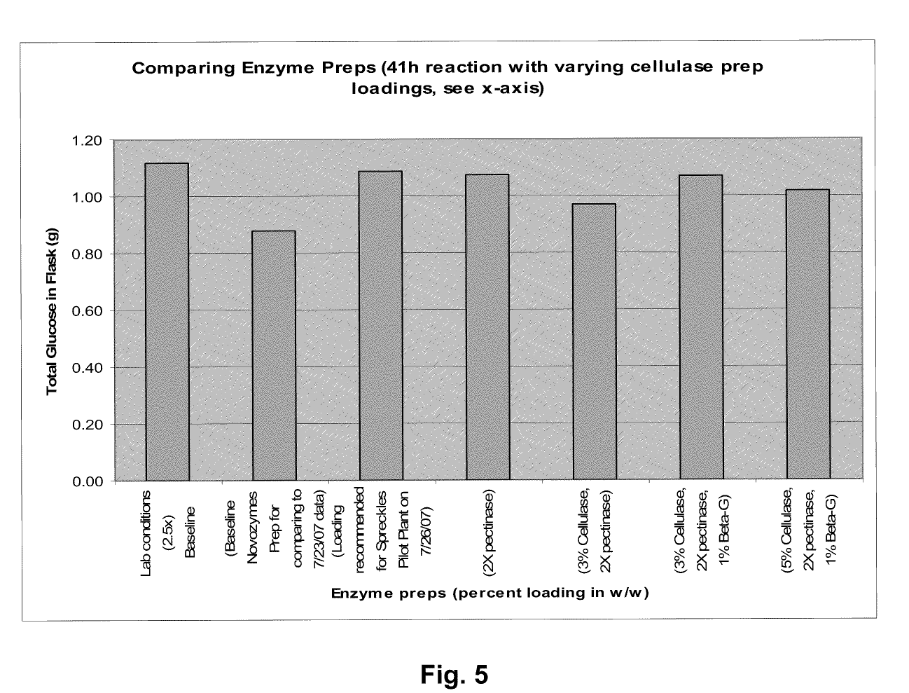

Laboratory tests were also conducted to optimize the enzymes, yeast, nutrients and antimicrobial agent additions. Enzymes were provided by Novozymes Corp. and tested for their efficacy and sugar yields. FIG. 5 illustrates the effect of enzyme addition on sugar extraction for various mixtures and dosages of enzymes.

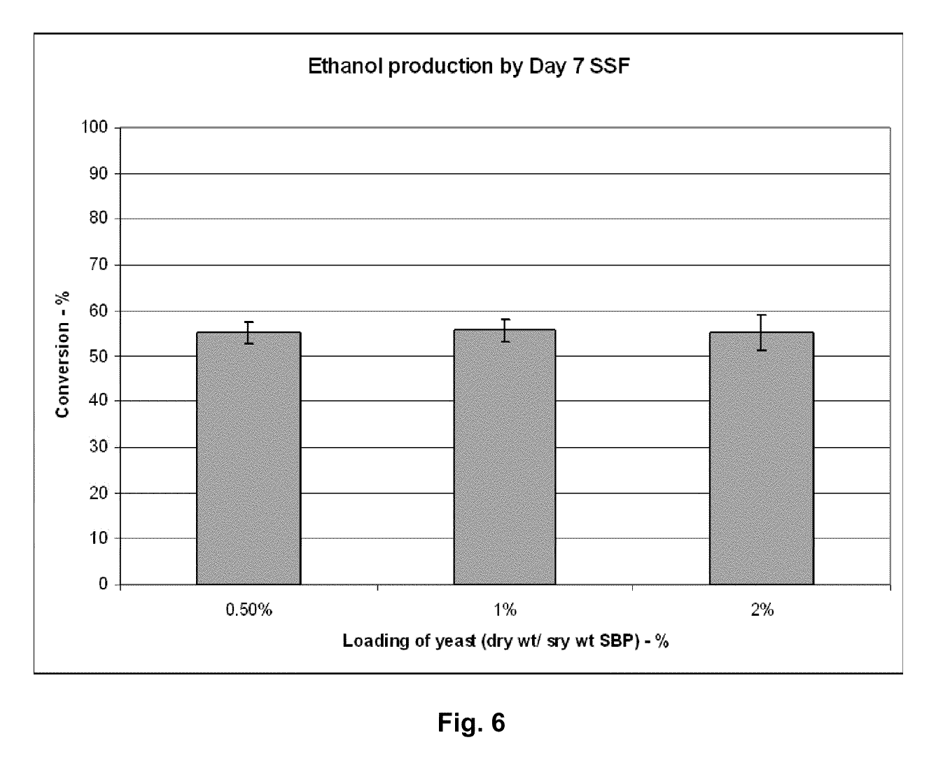

Commercial ethanol yeast was procured and tested for solid-state application. The products tested were all strains of Saccharomyces cerevisiae. The selected yeast, Ethanol Red™, was supplied by Fermentis®. Addition rates were optimized for ethanol yield. Commercially available yeast nutrients were tested at laboratory scale. FIG. 6 depicts the effect of yeast addition on ethanol production.

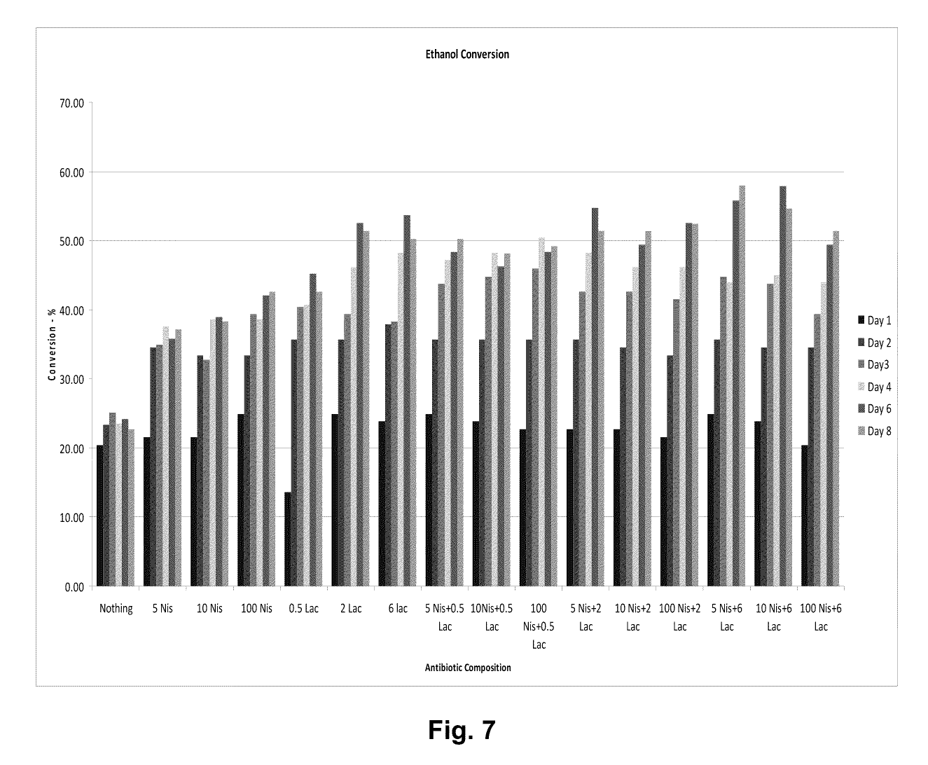

The performance of the antimicrobial agents Lactrol™ (virginiamycin) and Nisin™ was also tested in laboratory experiments. Addition rates for a test SSF heap were determined based on the results of these experiments and on the manufacturers' published recommendations. FIG. 7 shows the impact of varying antimicrobial agent dosage on ethanol production in a SSF bioreactor.

In another field test performed by Applicant, dried biomass in the form of sugar beet cossettes (sugar beets which have been sliced into french fry-like strips) was used as the initial feedstock. With cossettes, some of the sugars are already simple sugars and advantageously do not need the addition of enzymes for hydrolysis. The amount of simple sugars in sugar beets is typically between 10-20% of the total mass of the sugar beet. A fermentation agent is then necessary to convert these simple sugars to ethanol. The fermentation agent, typically yeast, requires the addition of nutrients for propagation. To suppress the proliferation of undesirable microorganisms that produce unwanted products and lower the ethanol yield, one or more antibiotic substances may be added.

In this case, Step 1 of FIG. 3 involved the loading of sugar beet cossettes into a suitable mixing vessel. Specifically, cossettes, containing 76% moisture as received, were loaded into an agricultural feed mixer by a front end loader. Approximately 4700 kg was charged for each batch. The mass was recorded by the mixer's load cell.

Step 3 of FIG. 3 was concerned with the sequential addition of reagent solutions other than enzymes. Specifically, reagent solutions were added sequentially in the following order: (a) yeast nutrients with antibiotics and (b) yeast. Solution volumes were measured by a totalizing flow meter.

Two batches of nutrient solution were produced for addition to the mixer. Each batch consisted of 327.4 g of Lactrol® (dissolved in 871 mL of ethanol and 1742 mL of water) and 1310 g of Fermaid K, added to 378.5 L of water. Dry components were added to 40° C. water and mixed for 15 minutes before the first addition to the mixer. Lactrol® was also added to the water in the solution tanks, so that microbial agents would not get into the system through the water addition, as well as maintaining the Lactrol® concentration throughout the test. 113.5 g of Lactrol® (dissolved in 455 mL of ethanol and 910 mL of water) was added to 18925 L of water in one of the solution tanks (used during the early stages of operation).

Two batches of yeast were made up for addition to the mixer. Each batch consisted of: 40.8 kg of yeast, 490 g of GO-FERN (suspended in one gallon of water), 1.64 g of Lactrol® (dissolved in a solution containing; 8 mL of ethanol and 16 mL of water), 164 g of Nisin™ (dissolved in a solution containing; 3 mL 12N Sulfuric acid and 1.63 L of water), added to 784 L of warm water. Dry components were pre-weighed and premixed, then added to water heated to 40° C. 4.085 kg of sucrose dissolved in 17 L of water was added to each batch and the batch was allowed to stand for 15 minutes before addition of the appropriate volume to the first batch in the mixer. This ensured the yeast population was active when added to the biomass solids.

Step 5 of FIG. 3 involved the addition of a bulking agent, such as screened almond shells, to the mixing vessel. Particularly, screened almond shells, containing 11.5% moisture as received, were loaded into the agricultural feed mixer by the front end loader. Approximately 1200 kg was charged for each batch. The mass was recorded by the mixer's load cell.

Step 6 of FIG. 3 dealt with the addition of water to the mixing vessel. Specifically, 1375 kg of water was added to achieve a saturated mix in the mixer. The quantity of water added was measured by both the mixer load cell and a flow meter in the supply line.

The mixing time for each batch was approximately 15 minutes. The total batch cycle time, including the time required to discharge the mixer, was 20 minutes. The field test quantities of each component in each batch and the totals for the heap are listed in the following table.

Using the measured quantities and analyses of the cossettes and almond shells, the theoretical ethanol yields were calculated. The field test resulted in 61% conversion of the available sugars into ethanol. The discrepancy with the weight-o-meter changed the yield by only a few percent. The field test results are illustrated in the following table.

Sucrose --> 4 EtOH + 4 CO₂

| | | |
|---|---|---|
| % Sucrose in Beets | 16.35% | |
| Theoretical Ethanol Production | 110.6 | L EtOH/ton cossette |
| Theoretical Ethanol Production | 8419.425 | L EtOH |
| EtOH in Tanks | 1482.166 | L EtOH |
| EtOH from condensate | 1 | L EtOH |
| EtOH from soak | 3615 | L EtOH |
| TOTAL EtOH | 5098 | L EtOH |
| % Yield | 61% | |

As noted above, the ration of biomass to bulking agent is important to maintaining adequate permeability in the static solid state bioreactor throughout the fermentation process. Darcy's law is often used to express the flow of liquid through a porous medium. A general form of the equation:

$$Q = - AK \frac{d h}{d l}$$

Q=total discharge (units m³/s)
K=hydraulic conductivity (units m/s)
A=cross-sectional area to the flow (units m²)

$$\frac{d h}{d l}$$

=is a change in hydraulic head Ah over the length L, limit of Δh as L goes to zero.

Hydraulic conductivity is related to permeability and when a fluid other than water at standard conditions is being used, the conductivity may be replaced by the permeability of the media. The two properties are related by:

K=kρg/μ=kg/v
k=permeability, (m²),
μ=fluid absolute viscosity, (N s/m²) and
v=fluid kinematic viscosity, (m²/s).
   Substitution of permeability for hydraulic conductivity back into Darcy's law yields:

$$Q = - A \frac{k ρ g}{μ} \frac{d h}{d l}$$

The hydraulic conductivity of the biomass to gas and liquid can thus be greatly increased by mixing a bulking agent with the biomass prior to loading into the static solid state bioreactor. The addition of a bulking agent helps maintain the hydraulic conductivity, counteracting the effects of compaction of the biomass under its own weight and breakdown of the biomass during conversion. The increased hydraulic conductivity eliminates channeling and also prevents the biomass from dramatically reducing in volume as the saccharification and/or fermentation processes occur. This prevents the biomass from pulling away from the walls of the bioreactor, another common cause of channeling.

Hydraulic conductivity is a key factor in the effectiveness of the temperature control means, namely the gas distribution system and the liquid distribution system, for the static solid state bioreactor. Adequate hydraulic conductivity is required to ensure that the flows of both gas and liquid can be maintained at the desired levels for the duration of the conversion process.

Bulking agents can be either degradable or non-degradable and can include, for example: sized aggregate, Styrofoam “peanuts” (preferably closed cell), plastic balls, almond shells and hulls, shredded tires, wood chips, and corn cobs. The selection of a bulking agent will depend on numerous factors including availability and also the type of biomass the bulking agent is to be mixed with. When selecting a bulking agent it is important to consider whether it will be inert with respect to the contents of the bioreactor or not. The influences of bulking agents that will somehow participate in the reactions taking place in the bioreactor must be accounted for.

Any bulking agent that when combined with the biomass, can pass the desired liquid and gas flows when under pressure, can be used. It is desired to maintain the ultimate hydraulic conductivity of the biomass to be greater than 10⁻⁵ cm/sec. More preferably the ultimate hydraulic conductivity of the biomass should be maintained greater than 10⁻⁴ cm/sec, which will generally limit the gas flow back-pressure to a desired maximum of less than 200 mm of water head. The ultimate hydraulic conductivity may be measured at the end of life, after the reactions in the bioreactor have finished. In this manner, it can be verified that the biomass bulking agent mixture maintain the necessary hydraulic conductivity throughout the life of the reaction in the bioreactor.

The quantity of bulking agent added will depend on the bulking agent particle size, size distribution, aspect ratio, shape, type and degradation rate. Table 5 lists some possible bulking agents (BA) to biomass (or feedstock) ratios that were found to have suitable hydraulic conductivity for processing in a static solid state bioreactor.

TABLE 5
Bulking Agent to Biomass Ratio

| | | *Bulking | | | | | |
|---|---|---|---|---|---|---|---|
| Bulking | | Agent | *Substrate | Ratio | | Column | |
| Agent | Substrate | (g) | (g) | (BA:BM) | | Size | Note |
| Plastic Balls | cardboard | 500 | 230 | 2.2 | 1 | 1 m | |
| Plastic Balls | cardboard | 250 | 450 | 0.6 | 1 | 1 m | |
| Plastic Balls | cardboard | 200 | 450 | 0.4 | 1 | 1 m | |
| Plastic Balls | cardboard | 200 | 400 | 0.5 | 1 | 1 m | |
| Plastic Balls | cardboard | 250 | 500 | 0.5 | 1 | 1 m | |
| Plastic Balls | cardboard | 450 | 900 | 0.5 | 1 | 3 m | |
| Tires | cardboard | 700 | 450 | 1.6 | 1 | 1 m | |
| Tires | cardboard | 584 | 450 | 1.3 | 1 | 1 m | |
| Tires | cardboard | 600 | 450 | 1.3 | 1 | 1 m | |
| Tires | cardboard | 400 | 450 | 0.9 | 1 | 1 m | |
| Tires | cardboard | 300 | 450 | 0.7 | 1 | 1 m | |
| Tires | cardboard | 1500 | 1800 | 0.8 | 1 | 3 m | |
| Tires | cardboard | 750 | 1800 | 0.4 | 1 | 3 m | |
| Tires | cardboard | 750 | 2000 | 0.4 | 1 | 3 m | |
| Plastic Balls | Sludge | 300 | 460 | 0.7 | 1 | 1 m | |
| Plastic Balls | Sludge | 300 | 500 | 0.6 | 1 | 1 m | |
| Packing | Sugar Beet | 5.46 | 750 | 0.00728 | 1 | BC-1 | or 1:1 by |
| Peanuts | Pulp | | | | | | volume |
| Almond Shells | Sugar Beet | 362 | 362 | 1 | 1 | BC-2 | |
| | Pulp | | | | | | |
| Almond Shells | Fresh Beets | 2000 | 8750 | 0.2 | 1 | BC-3 | |
| Almond Shells | Fresh Beets | 2000 | 8750 | 0.2 | 1 | BC-4 | |
| Packing | Fresh Beets | | | 0.5 | 1** | BC-5 | **Based on |
| Peanuts | | | | | | | volume |

*Note: Bulking Agent and Substrate weights are “as received”

Typical bulking agent to biomass mass ratios that have generally proven effective for use in a static solid state bioreactor such as that described in U.S. patent application Ser. No. 12/423,803 range from 1:5 to 1:1. The corresponding volume ratios will depend on the relative bulk densities of the biomass and bulking agent. Although larger ratios of bulking agent to biomass will tend to have better hydraulic conductivity for any given system, increased use of bulking agent will result in reduced volume of biomass that can be placed in the reactor.

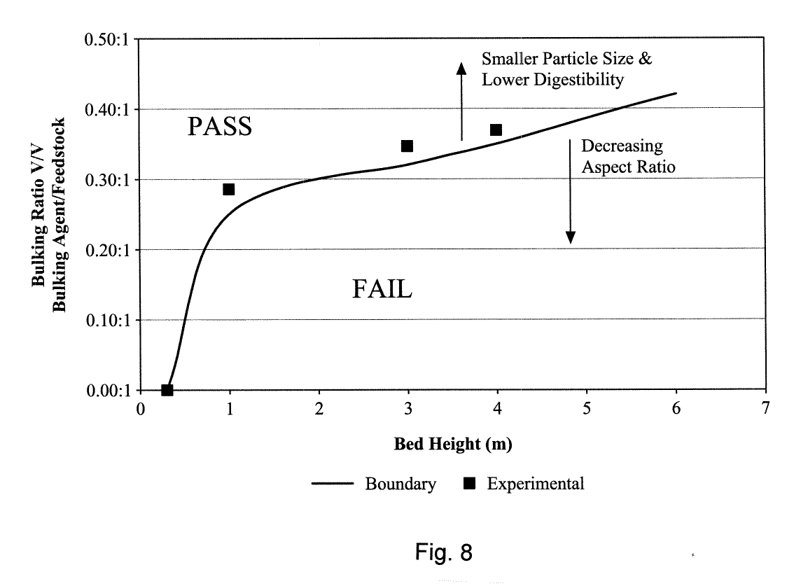

As noted above, the bulking agent to biomass (or feedstock) volume ratio influences the permeability in solid state fermentation. The graph in FIG. 8 shows the effect of bulking agent volume ratio to acceptable bed height in fermentation of waste paper based on irrigation rate for the experimental data in Table 6 below. As FIG. 8 shows, increasing the bed height of the solid state bioreactor requires an increased bulking agent to substrate volume ratio because of the increased bed self-weight. In FIG. 8, “Pass” and “Fail” refers to the hydraulic conductivity of the feedstock bed in the SSF reactor. In other words, it is considered to pass if liquid and gas can flow freely through bulked feedstock. The minimum acceptable “pass” irrigation rate for a given bed height is given in Table 2 and generally increases with bed height due to the increased volume and thus increased irrigation rates that are required to maintain the bed within an acceptable process temperature range.

TABLE 6
Effect of Bulking Agent Ratio on Acceptable Bed Height

| | | Weight Ratio | Volume Ratio | SSSF Ht | Irr. Rate |
|---|---|---|---|---|---|
| Bulking Agent | Substrate | (BA:BM) | (BA:BM) | (m) | (L/m²/h) |
| Plastic Balls | Waste Paper | 0.00:1 | 0.00:1 | 0.3 | 5 |
| Plastic Balls | Waste Paper | 0.44:1 | 0.29:1 | 1 | 5 |
| Plastic Balls | Waste Paper | 0.50:1 | 0.35:1 | 3 | 30 |
| Packing Peanuts | Waste Paper | 0.00:1 | 0.37:1 | 4 | 30 |

Preparing similar tables for other bulking material/feedstock systems will show that the “pass/fail” curve shown in FIG. 8 will shift as illustrated depending on a number of parameters. For example, decreasing feedstock particle size requires a higher bulking agent ratio due to the lower void volume and lower coefficient of permeability of the feedstock. Likewise, feedstocks with high aspect ratios (flat as opposed to round) also require a higher bulking agent ratio. On the other hand, feedstocks that digest completely tend to require a lower bulking agent ratio as the bed voidage increases as the reaction proceeds.

For any given system and reactor bed height, it is desirable to operate as close as possible to the boundary line shown in FIG. 8 in order to maximize the volume of the biomass feedstock that can be included in the bioreactor. Accordingly, the volume of the employed biomass is preferably less than 20%, and more preferably less than 10%, greater than that required by the boundary line for a given material system and bed height.

Although FIG. 8 has been prepared based on irrigation rate, a similar Pass/Fail curve may be prepared based on acceptable “pass” gas flow rates for a given bed height and material system.

While one or more embodiments have been described in connection with the figures hereinabove, the invention is not limited to these embodiments, but rather can be modified and adapted as appropriate. Thus, it is to be clearly understood that the above description was made only for purposes of an example and not as a limitation on the scope of the invention as claimed herein below.

What is claimed:

1. A method of preparing biomass for biotreatment in a static solid state bioreactor, the method comprising the steps of:
pre-mixing biomass with at least one biotreatment reagent;
adding a bulking agent to the pre-mixed biomass after a time sufficient for the at least one biotreatment reagent to have reacted with the biomass; and
mixing the added bulking agent with the pre-mixed biomass to homogenize the mixture prior to forming a static solid state bioreactor.

2. A method of preparing biomass for biotreatment in a static solid state bioreactor, the method comprising the steps of:
mixing biomass with at least one biodegradation reagent to form a first mixture;
pre-mixing a bulking agent with at least one additional biodegradation reagent; and
mixing the pre-mixed bulking agent with the formed first mixture to prepare a second mixture, the prepared second mixture being used to form a static solid state bioreactor.

3. A method of preparing biomass for biotreatment in a static solid state bioreactor, the method comprising the steps of:
pre-mixing biomass with at least one biotreatment reagent to prepare a first mixture;
pre-mixing a bulking agent with at least one additional biotreatment reagent;
adding the pre-mixed bulking agent to the first mixture after a time sufficient for the at least one biotreatment reagent to have reacted with the biomass; and
mixing the added pre-mixed bulking agent with the first mixture to prepare a second mixture for use in forming a static solid state bioreactor, the prepared second mixture having a substantially uniform distribution of bulking agent and biomass solids.

4. A method of preparing biomass for biotreatment in a static solid state bioreactor, the method comprising the steps of:
loading dried biomass into a mixing vessel;
rehydrating the dried biomass load in the mixing vessel;
adding a plurality of reagent solutions sequentially to the rehydrated biomass in the mixing vessel;
providing sufficient mixing time for the reagent solutions and the rehydrated biomass in the mixing vessel;
adding at least one bulking agent to the mixing vessel; and
adding water to the mixing vessel to attain a target hydration level of mixed biomass and bulking agent solids.

5. The method of claim 4, wherein the dried biomass is in the form of dried sugar beet pulp (SBP).

6. The method of claim 4, wherein the dried biomass is in the form of dried sugar beet cossettes.

7. The method of claim 1, wherein the bulking agent includes at least one organic material.

8. The method of claim 1, wherein the bulking agent includes at least one inorganic material.

9. The method of claim 1, wherein the bulking agent is selected from the group consisting of almond shells (screened and unscreened) and hulls, wood chips (bark and/or wood), beet chunks, corn cobs, corn stover, orange rinds, and wheat and rice straw.

10. The method of claim 1, wherein the bulking agent is selected from the group consisting of plastic balls (spheres, bioballs), styrofoam peanuts, shredded tires, and rocks.

11. The method of claim 1, wherein the biomass is selected from the group consisting of corn stover, corn fibers, wheat straw, wood wastes, urban wastes, switchgrass, rice straw, sugar beet pulp, citrus peels, and/or sugarcane bagasse.

12. The method of claim 1, wherein the at least one biotreatment reagent is selected from the group consisting of enzymes, antibiotics, yeast nutrients, yeast, water, and recycled solution.

13. The method of claim 1, wherein the at least one biotreatment reagent includes a fermentation agent.

14. The method of claim 4, wherein the mixing vessel is an agricultural feed mixer.

15. The method of claim 4, wherein the mixing vessel is a rotating drum.

16. The method of claim 4, wherein the mixing vessel is a screw mixer.

17. The method of claim 4, wherein the mixing vessel is a vertical-auger batch mixer.

18. The method of claim 1, wherein the static solid state bioreactor is a solid-state fermentation (SSF) bioreactor.

19. The method of claim 4, wherein the plurality of reagent solutions includes one or more enzymes.

20. The method of claim 19, wherein at least one of the enzymes is cellulase.

21. The method of claim 19, wherein at least one of the enzymes is beta-glucosidase.

22. The method of claim 19, wherein at least one of the enzymes is pectinase.

23. The method of claim 19, wherein at least one of the enzymes is Exoglucanase 1.

24. The method of claim 19, wherein at least one of the enzymes is Exoglucanase 2.

25. The method of claim 19, wherein at least one of the enzymes is Endoglucanase E1.

26. The method of claim 4, wherein the plurality of reagent solutions includes cellulase, beta-glucosidase and pectinase.

27. The method of claim 4, wherein the plurality of reagent solutions is selected from the group consisting of cellulase, beta-glucosidase, pectinase, Exoglucanase 1, Exoglucanase 2, and Endoglucanase E1.

28. A system for preparing biomass for biotreatment in a static solid state bioreactor, the system comprising:
a first stage which includes pre-mixing biomass with at least one biotreatment reagent; and
a second stage which includes the addition of a bulking agent to the pre-mixed biomass after a time sufficient for the at least one biotreatment reagent to have reacted with the biomass, the second stage further including mixing the added bulking agent with the pre-mixed biomass to homogenize the mixture prior to forming a static solid state bioreactor.

29. A system for preparing biomass for biotreatment in a static solid state bioreactor, the system comprising:
a first stage which includes mixing biomass with at least one biodegradation reagent to form a first mixture; and
a second stage which includes pre-mixing of a bulking agent with at least one additional biodegradation reagent, the second stage further including mixing the pre-mixed bulking agent with the formed first mixture to prepare a second mixture, the prepared second mixture being used to form a static solid state bioreactor.

30. A system for preparing biomass for biotreatment in a static solid state bioreactor, the system comprising:
a first stage which includes pre-mixing biomass with at least one biotreatment reagent to prepare a first mixture; and
a second stage comprising:
pre-mixing a bulking agent with at least one additional biotreatment reagent;
adding the pre-mixed bulking agent to the first mixture after a time sufficient for the at least one biotreatment reagent to have reacted with the biomass; and
mixing the added pre-mixed bulking agent with the first mixture to prepare a second mixture for use in forming a static solid state bioreactor, the prepared second mixture having a substantially uniform distribution of bulking agent and biomass solids.

31. The system of claim 28, wherein the biomass is pre-mixed with the at least one biotreatment reagent in a first mixing vessel.

32. The system of claim 31, wherein the added bulking agent is mixed with the pre-mixed biomass in a second mixing vessel.

33. The method of claim 4, wherein the plurality of reagent solutions excludes enzymes.

34. The system of claim 28, wherein the first and second stages are carried out in one mixing vessel.
